(19) United States
(12) Patent Application Publication
(10) Pub. No.: US 2012/0141410 A1
(43) Pub. Date: Jun. 7, 2012

(54) METHOD AND COMPOSITION FOR THE TREATMENT OF MODERATE TO SEVERE KERATOCONJUNCTIVITIS SICCA

(75) Inventor: Habib Torfi, Newport Beach, CA (US)

(73) Assignee: INVITRX, INC., Irvine, CA (US)

(21) Appl. No.: 13/230,476

(22) Filed: Sep. 12, 2011

Related U.S. Application Data
(63) Continuation of application No. PCT/US2010/025842, filed on Mar. 2, 2010.
(60) Provisional application No. 61/159,317, filed on Mar. 11, 2009.

(51) Int. Cl. A61K 38/20; A61P 27/02; A61K 38/19; A61K 38/27; A61K 38/17; A61K 38/18

(52) U.S. Cl. 424/85.2; 514/20.8; 514/7.6; 514/9.6; 514/8.9; 424/85.1; 514/9.1; 514/8.2; 514/11.4

(57) ABSTRACT

Embodiments of the invention relate to compositions and methods of dry eye or keratoconjunctivitis sicca.

This application claims priority to U.S. Provisional patent application No. 61/159,317, filed Mar. 11, 2009.

### FIELD OF THE INVENTION

This invention relates to the treatment of Keratoconjunctivitis Sicca and ophthalmic compositions useful in said treatment.

### BACKGROUND

It has now been shown that the classic aqueous-dominated tear film model of dry eye has been replaced by the more probable concept of a mucin-dominated gel. This gel has its highest concentration of mucin at the epithelial surfaces of the cornea and conjunctiva, and the mucin concentration gradually decreases farther out into the tear film. In this model, the presence of mucin remains significant for the structure, stability and function of the entire tear film. Recent studies of the tear film using laser interferometry and confocal microscopy indicates that the human tear film is 30 to 40 microns thick, more than four times thicker than earlier estimates (see for example Prydal, et al. 1992 and Prydal, et al. 2005). Based on tear film physiology and clinical observations, tear film abnormalities are commonly designated by focus on a specific deficiency, such as an aqueous tear deficiency, keratoconjunctivitis sicca (KCS), a mucin deficiency, a lipid abnormality, an impaired lid function, or an epitheliopathy. Although clinically useful, the simplistic concept of a lack of one component of the tear film as the cause of dry eye has given way to a much more sophisticated view of ocular surface disease that involves: (1) the health and regulation of the various glands contributing secretions to the tear film, (2) changes in the tear film itself, such as in osmolality and content of inflammatory mediators, and (3) what is viewed as a sort of “final common pathway”, the subsequent changes to the ocular surface (See McKenzie, et al. 2000). In fact, many clinicians and authors prefer the term “ocular surface disease” over “dry eye”, for it is change to the ocular surface, whatever the original cause, that results in the significant signs and symptoms of dry eye. The discomfort of ocular surface disease is expressed in ocular symptoms, such as dryness, grittiness, burning, soreness or scratchiness, with variation among individuals. These symptoms can also be exacerbated by factors such as environmental conditions and contact lens wear. The combination of varying clinical signs and symptoms has also been termed dry eye syndrome.

In the human eye, the secretory mucins MUC2 and MUCSAC have been detected (via transcripts at the nucleic acid level) from conjunctival isolates, and only MUCSAC has been localized to conjunctival goblet cells (See Pflugfelder, et al. 2000 and Sylvester, et al. 2001). Unique characteristics of normal human secreted ocular mucins are their wide size range and short oligosaccharide side chains.

The transmembrane mucin MUC1 is associated with the cell membranes of the entire corneal and conjunctival epithelial surface, except the goblet cells. Another transmembrane mucin is the mucin MUC4, which is associated with the cell membranes of the entire conjunctival epithelial surface, except the goblet cells.

In a mild to moderate dry eye, the goblet cell density is not significantly reduced, indicating that MUCSAC is most likely to be produced normally, in quantities sufficient to be spread over the entire ocular surface. However, localized early ocular surface changes resulting from dryness, such as that revealed by fluorescein or rose bengal staining, can be seen in the epithelia of the corneal and conjunctival surfaces. This localized damage to the ocular surface indicates that even marginal dryness might have a significant effect on the presence of functional MUC1 on the surface of the ocular epithelium. Since one of the proposed functions of MUC1 is to help the other, more abundant gel-forming ocular mucins adhere to the ocular surface, a paucity of MUC1 might significantly affect the stability of the tear film, even in the presence of an abundance of MUCSAC secreted by the conjunctival goblet cells (See Watanabe, et al. 2002 and Gipson 2004). There is some early evidence that with the progression of changes to the ocular surface mucins associated with dry eye, as detected by immunohistochemical methods, the goblet cells themselves try to make up for the lack of normal expression of MUC1 by the rest (non goblet cells) of the corneal and conjunctival surface epithelium, and begin expressing a MUC1-like molecule in their secretions.

The secreted ocular mucins are relatively large molecules, and have a significant role in the gel-forming nature of the tear film. The model of the greater part of the tear film being a highly hydrated mucus gel, rather than simply a watery aqueous layer, is becoming increasingly accepted. The viscoelasticity of the tear film derives from the specific structure and gel-forming properties of the ocular mucins, and allows the tear film to absorb the shear force of the blink, which would otherwise irritate and damage the ocular surface. The transmembrane mucin, on the other hand, serves more as a protective layer on the actual cellular surface of the ocular epithelium, functioning to directly protect and lubricate the ocular surface, as well as to anchor the highly hydrated gel (mucus) of the tear film gel-forming mucins, thereby assisting in the spreading and stability of the tear film over the ocular surface.

The importance of mucin in the natural tear fluid as a wetting agent, viscoelastic gel former, lubricant and barrier to bacterial adhesion has largely been reported. Limited success with so many various synthetic and substitute polymers indicate that supplementing the tear fluid with a compatible mucin from an exogenous source would appear to be a more direct and preferred method for addressing dry eye conditions. Part of the problem in the development of ocular surface changes in dry eye disease may be the dehydration of the mucus gel and subsequently the mucin layer of the cellular surface. Supplementing the tear fluid with mucin in an aqueous solution would be expected to help maintain the natural surface mucin layer of the eye by both the addition of the additional mucin molecules and the hydration provided by the aqueous vehicle.

The belief that the tear film is aqueous based and the ocular surface changes seen in Sjögren's syndrome are due to desiccation, cause eye care practitioners to water the dry eye. However, studies show that, as stated above, the tear film is dominated by mucin and not water. (See Nelson, et al. 1992) The human tear film is not a 7-10 μm thin film, but a 30-35 μm thick mucin gel. Bicarbonate may be critical to forming this gel as it is in forming the bicarbonate mucin gel that protects the stomach from autodigestion. (See Ubels, et al. 1995) The hallmark of the aqueous deficient dry eye, rose bengal staining of the conjunctiva, is not produced by desiccated cells, but is due to a deficiency in the protective mucin gel. (See Gilbard, et al. 1992) The ocular surface changes in dry eye include conjunctival squamous metaplasia, loss of integrity of cell membranes and junctional structures (fluorescein staining), and loss of the integrity of the mucin layer (rose bengal staining) Rose bengal staining and squamous metaplasia are not improved by the frequent application of non-preserved preparations. (See Nelson 1998) Bicarbonate and electrolyte solutions promote recovery of barrier function and ultrastructure in damaged ocular surface cells and increase corneal glycogen and goblet cell density. (See VanSetten 1990 and Kiatazawa, et al. 1990, respectively) These solutions, however, do not totally reverse ocular surface disease seen in Sjögren's syndrome. Even with the addition of electrolytes and bicarbonate to artificial tears, watering the dry eye is not enough.

It has been found that the application of autologous serum improved fluorescein and rose bengal scores and squamous metaplasia. This treatment also resulted in significant upregulation of MUC-1 in conjunctival epithelial cell cultures. The authors believed that the epidermal growth factor (EGF), vitamin A, and transforming growth factorβ (TGF-β) found in serum represent critical components missing from the tears of patients with Sjögren's syndrome.

Studies have shown that some cytokines play an important role in the regulation of proliferation, differentiation, and maturation of the ocular surface epithelium, while the cytokines may be harmful. (See Weng, et al. 1996) Experimental studies demonstrate that EGF and hepatocyte growth factor (HGF), (See Vervo, et al. 1997; Van Sletten 1996; Yoshino, et al. 1996; Slomiany, et al. 1991; and Sotozono, et al. 1998) which are present in human tears and secreted by the lacrimal gland, are important in corneal wound healing. Both also increase as aqueous tear production increases. TGF-α and TGF-β are found in human tears. (See Ubels, et al. 1986 and Ono, et al. 1994) Both are probably involved in corneal epithelial cell growth and differentiation. (See Ono, et al. 1994) Retinol, also secreted by the lacrimal gland and found in the tear film, is necessary for the maintenance of healthy ocular surface epithelium. (See Ono, et al. 1994) Not only may the tear film of patients with Sjögren's syndrome be missing critical components, tears may actually contain substances that lead to ocular surface injury. Cytokines may be produced in or by the lacrimal gland in response to inflammation. These factors, delivered to the ocular surface by the tear fluid, may lead to inflammation of the ocular surface. mRNA for interleukins IL-1 and IL-6 has been detected in the lacrimal glands of autoimmune female MRL/lpr mice. (See Wilson, et al. 1996) Increased levels of IL-1 induces keratocyte apoptosis and metalloproteinases. (See Wilson, et al. 1996 and Girard, et al. 1991) IL-6 induces lymphocytic differentiation.

In Sjögren's syndrome, reflex tearing decreases with increased lymphocytic infiltration of the lacrimal gland. (See Tsubota, et al. 1996) Reflex tearing flushes debris from the ocular surface, dilutes substances in the tear film, and delivers higher amounts of certain cytokines to the ocular surface. The loss of reflex tearing results in reduced tear clearance causing prolonged retention of substances in the tear film. (See Barton, et al. 1997) It is likely that the loss of reflex tearing also results in the lack of delivery of cytokines and retinol critical to the growth and differentiation of ocular surface epithelial cells.

The upregulation of MUC-1 suggests there are substances in serum, which promote reformation of the mucin gel, and, therefore, resolution of rose bengal staining. It is believed that similar substances, that are important in the maintenance of the mucin gel, are probably missing in the Sjögren's dry eye.

Others have speculated on the use of serum tears. (See Fox, et al. 1984) Tsubota et al suggests that serum tears, alone, may not be sufficient to treat dry eye. For example it has been found that the presence of cytokines and retinol are critical for the growth, differentiation, and wound healing of the ocular surface. Artificial tears flush out debris; dilute substances trapped in the tear film, and increase tear clearance. They do not, however, provide all the factors critical for the maintenance and repair of the ocular surface.

### SUMMARY OF THE INVENTION

The present invention provides a method of treating dry eye by topically administering to the eye a human conjunctiva-derived mucin in an ocular drop instillable composition which derived mucin is similar to those of the transmembrane mucin expressed on the ocular surface epithelium, and to the gel-forming mucins secreted by the goblet cells. The novel compositions of this invention protect the ocular surface from dryness and absorb shear forces of the blink, and assist the eye's own secreted gel forming mucins (predominantly MUC5) in maintaining their viscoelastic properties and ensuing structure and stability of the tear film, thereby slowing or preventing the changes to the ocular surface seen in dry eye conditions.

In one aspect of this invention, there is disclosed, a topical ophthalmic composition for treating and/or preventing dry eye or keratoconjunctivitis sicca (KCS) in accordance with the preferred embodiments of the present invention. The topical ophthalmic composition comprises a conditioned medium or extract or concentrate thereof, wherein the conditioned medium is generated by incubating a nutrient medium with cornea and/or conjunctiva cells under conditions adapted to promote secretion of at least one growth factor into the nutrient medium, wherein said growth factor is present in the conditioned medium or extract or concentrate thereof in an amount which is therapeutically effective in treating and/or preventing dry eye or KCS.

In preferred embodiments, the topical ophthalmic composition further comprises a thickener. In some embodiments, the thickener can comprise a biocompatible or biodegradable polymer, including but not limited to, polysaccharides and polyesters (for example, see Sigma Aldrich for commercially available biocompatible polymers. Preferably, the polysaccharide can comprise a cellulosic material, such as carboxymethylcellulose.

In another preferred embodiment, the topical ophthalmic composition further comprises purified water, electrolytes, and/or at least one preservative.

### DETAILED DESCRIPTION OF THE PREFERRED EMBODIMENT

In preferred embodiments, the present invention relates to topical therapeutic and/or prophylactic formulations for treating dry eye or KCS, comprising a conditioned medium from corneal cell cultures. The cells are preferably human to reduce the risk of an immune response. In preferred embodiments of the present invention, cultures of primary human corneal cells are used to condition the nutrient medium in which they are bathed. In another embodiment, the conditioned medium is from corneal stem cells, or corneal cells differentiated from stem cells or other types of pluripotent or multipotent cells. Medium conditioned by such cell cultures contain a variety of naturally secreted proteins, including biologically active growth factors.

Growth factors, such as transforming growth factor-β, also known in the art as TGF-β, are induced by certain stress proteins during wound healing. Two known stress proteins are GRP78 and HSP90. These proteins stabilize cellular structures and render the cells resistant to adverse conditions. The TGF-β family of dimeric proteins includes TGF-β1, TGF-β2, and TGF-β3 and regulates the growth and differentiation of many cell types. Furthermore, this family of proteins exhibits a range of biological effects, stimulating the growth of some cell types and inhibiting the growth of other cell types. TGF-β has also been shown to increase the expression of extracellular matrix proteins including collagen and fibronectin and to accelerate the healing of wounds.

The growth factors that are derived from the above cell cultures may include, but are not limited to: GM-CSF; IL-15; IL-1a; IL-2; IL-4; IL-5; IL-6; IL-7; IL-8; MCP-1; TNF_{α}; FGF-2; Flt-3; PDGF-AA; TGF-beta1; TGF-beta2; and TGF-beta3. In some embodiments, the conditioned medium or ophthalmic composition comprises one or more of these growth factors, wherein the growth factors may be present in a concentration (each growth factor may be present in different concentrations): from about 0.1 pg/mL to about 10 pg/mL; from about 1 pg/mL to about 1 ng/mL; from about 1 ng/mL to about 1 μg/mL; or from about 1 μg/mL to about 1 mg/mL. As used herein, the term “about” with respect to growth factor concentrations can mean a variance of 10% of the concentration. For example, about 0.1 pg/mL can mean 0.1 pg/mL±0.01 pg/mL, or about 1 μg/mL can mean 1 μg/mL±0.1 μg/mL.

In some embodiments, the topical therapeutic and/or prophylactic formulations for treating dry eye or KCS further comprises one or more of the following growth factors present in said concentrations: about 0.6 pg/mL of GM-CSF, about 0.2 pg/mL of IL-15, about 0.3 pg/mL of IL-1a, about pg/mL of 1.6 IL-2, about pg/mL of 0.6 IL-4, about 2.8 pg/mL of IL-6, about 0.1 pg/mL of IL-7, about 0.3 pg/mL of IL-8, about 2.3 pg/mL of MCP-1, about 0.1 pg/mL of TNFα, about 4 pg/mL of FGF-2, about 2 pg/mL of Flt-3, about 16 pg/mL of PDGF-AA, about 1035 pg/mL of TGF-β1, about 46 pg/mL of TGF-β3, and about 130 pg/mL of TGF-β2.

The cells may be readily isolated by disaggregating an appropriate eye or tissue which is to serve as the source of the cells. This may be readily accomplished using techniques known to those skilled in the art. For example, the tissue can be disaggregated mechanically and/or treated with digestive enzymes and/or chelating agents that weaken the connections between neighboring cells making it possible to disperse the tissue into a suspension of individual cells without appreciable cell breakage. Enzymatic dissociation can be accomplished by mincing the tissue and treating the minced tissue with any of a number of digestive enzymes either alone or in combination. These include but are not limited to trypsin, chymotrypsin, collagenase, elastase, and/or hyaluronidase, DNase, pronase, dispase etc. Mechanical disruption can also be accomplished by a number of methods including, but not limited to, the use of grinders, blenders, sieves, homogenizers, pressure cells, or insonators to name but a few. For a review of tissue disaggregation techniques, see Freshney, Culture of Animal Cells: A Manual of Basic Technique, 2d Ed., A. R. Liss, Inc., New York, 1987, Ch. 9, pp. 107-126, which is hereby incorporated by reference in its entirety.

Once the tissue has been reduced to a suspension of individual cells, the suspension can be fractionated into subpopulations from which the cells and/or elements can be obtained. This also may be accomplished using standard techniques for cell separation including, but not limited to, cloning and selection of specific cell types, selective destruction of unwanted cells (negative selection), separation based upon differential cell agglutinability in the mixed population, freeze-thaw procedures, differential adherence properties of the cells in the mixed population, filtration, conventional and zonal centrifugation, centrifugal elutriation (counterstreaming centrifugation), unit gravity separation, countercurrent distribution, electrophoresis and fluorescence-activated cell sorting. For a review of clonal selection and cell separation techniques, see Freshney, Culture of Animal Cells: A Manual of Basic Techniques, 2d Ed., A. R. Liss, Inc., New York, 1987, Ch. 11 and 12, pp. 137-168, which is hereby incorporated by reference in its entirety.

The cells utilized to prepare the conditioned medium utilized in the method of this invention can be cultured in accordance with preferred embodiments by any means known in the art, including those processes disclosed in U.S. Patent Application Ser. No. 60/853,402 filed Oct. 19, 2006, which is hereby incorporated by reference in its entirety.

In some embodiments of the present invention, the growth factor-rich conditioned media may be diluted, concentrated and/or preserved prior to combining it with the variety of formulations for topical application to the eye of a patient suffering from dry eye or KCS. Concentration may be accomplished by any conventional methods known in the art, including for example, freeze-drying, vacuum-drying, evaporation, etc. Moreover, particular growth factors may be concentrated by affinity chromatography or other conventional methods for protein/peptide purification. Dilution methods may include addition of deionized water. Preservation methods may include freeze-drying, spray-drying, foam-drying, etc. In a preferred embodiment, the medium is filtered with a 7 micron filter, then preservatives and other ingredients and/or supplements are added to the medium, and the medium is stored in a refrigerator. In addition, the conditioned medium may be subjected to further processing, e.g., affinity chromatography, to differentially concentrate or remove certain medium components.

Following removal of the cell conditioned medium, it may be necessary to further process the resulting supernatant. Such processing may include, but is not limited to, centrifugation, product isolation and purification, dilution of the media or concentration of the media by a water flux filtration device or by defiltration using the methods described in Cell & Tissue Culture: Laboratory Procedures, supra, pp 29 D:0.1-29D:0.4, which is hereby incorporated by reference in its entirety.

The conditioned medium may be further processed for product isolation and purification to remove unwanted components. The methods used for product isolation and purification so that optimal biological activity is maintained will be readily apparent to one of ordinary skill in the art. For example, it may be desirous to purify a growth factor, regulatory factor, etc. Such methods include, but are not limited to, gel chromatography (using matrices such as Sephadex) ion exchange, metal chelate affinity chromatography with an insoluble matrix such as cross-linked agarose, HPLC purification and hydrophobic interaction chromatography of the conditioned media. Such techniques are described in greater detail in Cell & Tissue Culture; Laboratory Procedures, supra. Of course, appropriate measures may be taken to maintain sterility. Alternatively, sterilization may be necessary and can be accomplished by methods known to one of ordinary skill in the art, such as, for example, heat and/or filter sterilization taking care to preserve the desired biological activity.

In a preferred embodiment, the media is filtered or centrifuged to prevent cell inclusion. It may then be diluted, e.g., with a phosphate buffer solution (PBS) or deionized water, if the growth factor concentrations are too high. Alternatively, the conditioned medium may be concentrated if the growth factor levels are not sufficiently high. The diluted or concentrated media may then be combined with an ophthalmically-acceptable vehicle, e.g. purified water, or an aqueous isotonic solution.

It should be understood that the following protocol is offered by way of example only and may be modified using methods known to those of skill in the relevant art. Moreover, this example is not to be construed as limiting the scope of the invention which is defined by the claims.

The active ingredient used in the method and compositions of the present invention is prepared as follows: Anterior segment of the eye is removed under sterile conditions by a circular incision through the sclera 2 mm below the limbus. The segment is carefully transferred into a dish containing Dulbecco's modified Eagles medium (DMEM), supplemented with 3% foetal calf serum (FCS), 50 mg/ml of gentamicin and 5 mg/ml of amphotericin B. The iris-ciliary body, lens, and corneal endothelium are microscopically removed. The specimen is dissected into three zones, i.e. the central cornea, peripheral cornea and limbus, and freed of any adhesive tissue fragments. 5 ml of 0.25% trypsin EDTA are added and the resulting mixture is incubated at 37° C. After incubation of 1 hr both the central and peripheral corneal specimens are centrifuged at 800×g for 10 min. The epithelial sheets are resuspended in 0.25% trypsin and EDTA for 10 minutes with intermittent gentle shaking. Enzymatic digestion is halted with the addition of DMEM containing 3% FCS. The isolated three cell types in are cultured both Epi-Life Media (Cascade Biologic) and DMEM containing 3% FCS and antibiotics. The cultures are incubated at 37° C., 95% humidity, and 5% CO₂. The media may be changed every 2 days to every 6 days, depending on type of media used. Cells are either further cultured or processed for freezing at 60-70% confluence.

The cells are formulated into a topical ophthalmic composition for use in the method of the invention as follows:

6.25 gm of CMC is blended with Ringers for 30-60 minutes. The resulting suspension is poured into non-sterile serum bottles. The bottles are capped and autoclaved for 20 minutes at 121 degree C.

The autoclaved suspension is combined with the cell supernate and put in 5 ml bottles.

In preferred embodiments, some of the growth factors secreted into the medium have the following concentrations (in picograms per mL):

| Growth | GM- | IL- | IL- | | | | | | | | |
|---|---|---|---|---|---|---|---|---|---|---|---|
| Factor | CSF | 15 | 1a | IL-2 | IL-4 | IL-5 | IL-6 | IL-7 | IL-8 | MCP-1 | TNFα |
| Pg/ml | 0.6 | 0.2 | 0.3 | 1.6 | 0.6 | 0.0 | 2.8 | 0.1 | 0.3 | 2.3 | 0.1 |

| | | | | | | PDGF- |
|---|---|---|---|---|---|---|
| Growth Factor | FGF-2 | VEGF | EGF | Flt-3 | PDGF-AA | AB/BB |
| Pg/ml | 4 | nd | nd | 2 | 16 | nd |

| Growth | | | |
|---|---|---|---|
| Factor | TGF-β1 | TGF-β3 | TGF-β2 |
| Pg/ml | 1035 | 46 | 130 |

In other embodiments, the conditioned medium or ophthalmic composition comprises one or more of the growth factors listed in the Tables above, wherein the growth factors are present in a concentration: from about 0.1 pg/mL to about 10 pg/mL; from about 1 pg/mL to about 1 ng/mL; from about 1 ng/mL to about 1 pg/mL; from about 1 μg/mL to about 1 mg/mL.

### Therapeutic Formulations

The conditioned medium may be formulated into a topical ophthalmic composition for preventing, reducing and/or eliminating dry eye or KCS.

In a preferred embodiment the conditioned cell medium is formulated as a drop, and/or serum for topical application, with or without additional growth factors, peptides, and/or other proteins and biologically active substances, including, but not limited to, those discussed herein.

In one preferred embodiment, the formulated topical ophthalmic composition combines therapeutically effective amounts of conditioned medium (or concentrates or extracts thereof) with a thickener, purified water, and at least one preservative.

In another preferred embodiment, the thickener comprises carboxymethylcellulose or methylcellulose, or polyvinylpyrrolidone or a polyacrylic acid polymer or copolymer.

Therapeutic products contained in the conditioned media include, but are not limited to, peptides, growth factors, enzymes, hormones, cytokines, antigens, antibodies, clotting factors, and regulatory proteins. Of course, the medium may be further processed to concentrate or reduce one or more factor or component contained within the medium, for example, enrichment of a growth factor using immunoaffinity chromatography or, conversely, removal of a less desirable component.

Assays commonly employed by those of skill in the art may be utilized to test the activity of the particular factor or factors, thereby ensuring that an acceptable level of biological activity (e.g., a therapeutically effective activity) is retained and/or generated by post-harvest processing. Doses of such therapeutic factors are well known to those of skill in the art and may be found in pharmaceutical compedia such as the PHYSICIANS DESK REFERENCE, Medical Economics Data Publishers; REMINGTON'S PHARMACEUTICAL SCIENCES, Mack Publishing Co.; GOODMAN & GILMAN, THE PHARMACOLOGICAL BASIS OF THERAPEUTICS, McGraw Hill Publ., THE CHEMOTHERAPY SOURCE BOOK, Williams and Wilkens Publishers.

The therapeutically effective doses of any of the growth factors, drugs or other active agents described above may routinely be determined using techniques well known to those of skill in the art. A “therapeutically effective” dose refers to that amount of the compound sufficient to result in amelioration of at least one symptom of dry eye or KCS.

Alternatively, the conditioned cell medium may be formulated with polymerizable or cross-linking hydrogels as described in U.S. Pat. Nos. 5,709,854; 5,516,532; 5,654,381; and WO 98/52543, each of which is incorporated herein by reference in its entirety. Examples of materials which can be used to form a hydrogel include modified alginates. Alginate is a carbohydrate polymer isolated from seaweed, which can be cross-linked to form a hydrogel by exposure to a divalent cation such as calcium, as described, for example in WO 94125080, the disclosure of which is incorporated herein by reference. Alginate is ionically cross-linked in the presence of divalent cations, in water, at room temperature, to form a hydrogel matrix. As used herein, the term “modified alginates” refers to chemically modified alginates with modified hydrogel properties.

Additionally, polysaccharides which gel by exposure to monovalent cations, including bacterial polysaccharides, such as gellan gum, and plant polysaccharides, such as carrageenans, may be cross-linked to form a hydrogel using methods analogous to those available for the cross-linking of alginates described above.

Modified hyaluronic acid derivatives may also be useful. As used herein, the term “hyaluronic acids” refers to natural and chemically modified hyaluronic acids. Modified hyaluronic acids may be designed and synthesized with preselected chemical modifications to adjust the rate and degree of cross-linking and biodegradation.

Covalently cross-linkable hydrogel precursors also are useful. For example, a water soluble polyamine, such as chitosan, can be cross-linked with a water soluble diisothiocyanate, such as polyethylene glycol diisothiocyanate.

Alternatively, polymers may be utilized which include substituents which are cross-linked by a radical reaction upon contact with a radical initiator, such as those disclosed in Naughton et al. U.S. Pat. No. 6,372,494; incorporated herein in its entirety by reference.

While a number of preferred embodiments of the invention and variations thereof have been described in detail, other modifications and methods of using and applications for the same will be apparent to those of skill in the art. Accordingly, it should be understood that various applications, modifications, materials, and substitutions may be made of equivalents without departing from the spirit of the invention or the scope of the claims. It should be understood that the invention is not limited to the embodiments set forth herein for purposes of exemplification, but is to be defined only by a fair reading of the appended claims, including the full range of equivalency to which each element thereof is entitled.

### LIST OF REFERENCES CITED HEREIN

Prydal J I, Artal P, Woon H, et al. Study of human precorneal tear film thickness and structure using laser interferometry. Invest Ophthalmol Vis Sci 1992; 33:2006-2011.
Prydal J, Campbell F. Study of precorneal fluid thickness and structure by interferometry and confocal microscopy. Invest Ophthalmol Vis Sci 1992; 33:1996-2005.
McKenzie R W, Jumblatt J E, Jumblatt M M. Quantification of MUC2 and MUC5AC transcripts in human conjunctiva. Invest Ophthalmol Vis Sci. 2000 March; 41(3):703-8.
Pflugfelder S C, Solomon A, Stern M E. The diagnosis and management of dry eye: a twenty-five-year review. Cornea. 2000 September; 19(5):644-9.
Watanabe H. Significance of mucin on the ocular surface. Cornea. 2002 March; 21(2 Suppl 1):517-22.
Gipson I K. Distribution of mucins at the ocular surface. Exp Eye Res. 2004 March; 78(3):379-88.
Berry M, Ellingham R B, Corfield A P. Human preocular mucins reflect changes in surface physiology. Br J Ophthalmol. 2004 March; 88(3):377-83.
Sylvester P A, Myerscough N, Warren B F, Carlstedt I, Corfield A P, Durdey P, Thomas M G. Differential expression of the chromosome 11 mucin genes in colorectal cancer. J Pathol. 2001 October; 195(3):327-35.
Nelson J D, Gordon J F. Topical fibronectin in the treatment of keratoconjunctivitis sicca. Chiron keratoconjunctivitis sicca study group. Am J Ophthalmol 1992; 114:441-447.
Ubels J, McCartney M, Lantz W, et al. Effects of preservative-free artificial tear solutions on corneal epithelial structure and function. Arch Ophthalmol 1995; 113:371-378.
Gilbard J P, Rossi S R. An electrolyte-based solution that increases corneal glycogen and conjunctival goblet-cell density in a rabbit model for keratoconjunctivitis sicca. Ophthalmology 1992; 99:600-604.
Nelson J. A clinician looks at the tear film. Adv Exp Med Biol 1998; 438:1-9.
Van Setten G. Epidermal growth factor in human tear fluid: increased release but decreased concentrations during reflex tearing. Curr Eye Res 1990; 9:79-83.
Kiatazawa T, Kinoshita S, Fujita K, et al. The mechanism of accelerated corneal epithelial healing by human epidermal growth factor. Invest Ophthalmol Vis Sci 1990; 31:1773-1778.
Ii Q, Weng J, Mohan R, et al. Hepatocyte growth factor and hepatocyte growth factor receptor in the lacrimal glands, tears, and cornea. Invest Ophthalmol Vis Sci 1996; 37:727-739.
Tervo T, Vesaluuoma M, Bennett G, et al. Tear hepatocyte growth factor (HGF) availability increases markedly after excimer laser surface ablation. Exp Eye Res 1997; 64:501-504.
Van Sletten G, Macauley S, Humphreys-Beher M, et al. Detection of transforming growth factor-alpha mRNA in rat lacrimal glands and characterization of transforming growth factor-alpha in human tears. Invest Ophthalmol Vis Sci 1996; 37:166-173.
Yoshino K, Rahul G, Monroy D, et al. Production and secretion of transforming growth factor beta (TGF-) by the human lacrimal gland. Curr Eye Res 1996; 15:615-624.
Slomiany B L, Slomiany A. Role of mucus in gastric mucosal protection. J Physiol Pharmacol 1991; 42:147-161.
Sotozono C, Kinoshita S. Growth factors and cytokines in corneal wound healing. In: Nishida T, ed. Proceedings: corneal healing responses to injuries and refractive surgeries. Amsterdam: Kugler Publications, 1998; 29-38.
Ubels J, Loley K, Rismondo V. Retinol secretion by the lacrimal gland. Invest Ophthalmol Vis Sci 1986; 27:1261-1269.
Ono M, Huang Z, Wickam L, et al. Analysis of androgen receptors and cytokines in lacrimal glands of a mouse model of Sjögren's syndrome. Invest Ophthalmol Vis Sci 1994; 35:S1793.
Wilson S, He Y, Weng J, et al. Epithelial injury induces keratocyte apoptosis: hypothesized role for interleukin-1 system in modulation of corneal tissue organization wound healing. Exp Eye Res 1996; 62:325-327.
Girard M, Matsubara M, Fine M. Transforming growth factor-beta and interleukin-1 modulate metalloproteinase expression in corneal stromal cells. Invest Ophthalmol Vis Sci 1991; 31:2441-2454.
Tsubota K, Xu K, Fujihara T, et al. Decreased reflex tearing is associated with lymphocytic infiltration in lacrimal glands. J Rheum 1996; 23:313-320.
Barton K, Monroy D, Nava A, et al. Inflammatory cytokines in the tears of patients with ocular rosacea. Ophthalmology 1997; 104:1868-1874.
Fox R, Chan R, Michelson J, et al. Beneficial effect of artificial tears made with autologous serum in patients with keratoconjunctivitis sicca. Arthritis Rheum 1984; 27:459-461.

What is claimed is:

1. A method for treating dry eye or keratonconjunctivitis sicca (KCS) comprising:
providing a therapeutic agent comprising a therapeutically effective amount of human conjunctive-derived mucin in an ophthalmic composition, said mucin being provided in combination with a pharmaceutically acceptable vehicle; and
administering said therapeutic agent topically to the ocular surface or immediate vicinity of an eye of a patient.

2. The method of claim 1 wherein in said administering step, said therapeutic agent is applied to the ocular surface of the eye.

3. The method of claim 1 wherein in said administering step, said therapeutic agent is applied to a region of the eye adjacent the ocular surface.

4. The method of claim 1 wherein in said providing step, said therapeutic agent further comprises one or more human growth factors.

5. The method of claim 1 wherein in said providing step, said therapeutic agent further comprises retinol.

6. The method of claim 4 wherein said human growth factors comprise EGF and TGF-beta.

7. The method of claim 4 wherein said human growth factors are selected from the group consisting of: GM-CSF; IL-15; IL-1a; IL-2; IL-4; IL-5; IL-6; IL-7; IL-8; MCP-1; TNFα; FGF-2; Flt-3; PDGF-AA; TGF-beta1; TGF-beta2; and TGF-beta3

8. The method of claim 4 wherein said ophthalmic composition comprises bicarbonate.

9. The method of claim 4 wherein said therapeutic agent further comprises about 0.6 pg/mL of GM-CSF, about 0.2 pg/mL of IL-15, about 0.3 pg/mL of IL-1a, about pg/mL of 1.6 IL-2, about pg/mL of 0.6 IL-4, about 2.8 pg/mL of IL-6, about 0.1 pg/mL of IL-7, about 0.3 pg/mL of IL-8, about 2.3 pg/mL of MCP-1, about 0.1 pg/mL of TNFα, about 4 pg/mL of FGF-2, about 2 pg/mL of Flt-3, about 16 pg/mL of PDGF-AA, about 1035 pg/mL of TGF-β1, about 46 pg/mL of TGF-β3, and about 130 pg/mL of TGF-β2.

10. A topical ophthalmic composition for treating and/or preventing dry eye, comprising a conditioned medium or extract or concentrate thereof, wherein said conditioned medium is generated by incubating a nutrient medium with substantially human conjunctiva cells under conditions adapted to promote secretion of at least one human growth hormone into the nutrient medium, wherein at least one human growth hormone is present in said conditioned medium or extract or concentrate thereof in an amount sufficient to treat or prevent dry eye.

11. The topical ophthalmic composition of claim 10, further comprising a pharmaceutically-acceptable vehicle.

12. The topical ophthalmic composition of claim 11, further comprising a thickener wherein said thickener comprises carboxymethylcellulose.

13. The topical ophthalmic composition of claim 11, wherein said pharmaceutically-acceptable vehicle comprises purified water.

14. A topical ophthalmic composition for treating or preventing dry eye or keratoconjunctivitis which comprises a therapeutic agent derived from a conditioned medium, or extract or concentrate thereof, wherein said conditioned medium is generated by incubating a nutrient medium with substantially human conjunctiva cells under conditions adapted to promote secretion of at least one human growth hormone into the nutrient medium, wherein at least one human growth hormone is present in said conditioned medium or extract or concentrate thereof in an amount sufficient to treat or prevent dry eye.

15. The composition of claim 14 further comprising retinol.

16. The composition of claim 14 wherein said therapeutic agent further comprises one or more human growth factors.

17. The composition of claim 16 wherein said growth factors are selected from the group consisting of: GM-CSF, IL-15, IL-1a, IL-2, IL-4, IL-5, IL-6, IL-7, IL-8, MCP-1, TNFα, FGF-2, Flt-3, PDGF-AA, TGF-beta1, TGF-beta2, and TGF-beta3.

18. The composition of claim 17 further comprising bicarbonate.

19. The composition of claim 14 wherein said therapeutic agent further comprises about 0.6 pg/mL of GM-CSF, about 0.2 pg/mL of IL-15, about 0.3 pg/mL of IL-1a, about pg/mL of 1.6 IL-2, about pg/mL of 0.6 IL-4, about 2.8 pg/mL of IL-6, about 0.1 pg/mL of IL-7, about 0.3 pg/mL of IL-8, about 2.3 pg/mL of MCP-1, about 0.1 pg/mL of TNFα, about 4 pg/mL of FGF-2, about 2 pg/mL of Flt-3, about 16 pg/mL of PDGF-AA, about 1035 pg/mL of TGF-β1, about 46 pg/mL of TGF-β3, and about 130 pg/mL of TGF-β2.
